(19) United States
(12) Patent Application Publication
(10) Pub. No.: US 2013/0052310 A1
(43) Pub. Date: Feb. 28, 2013

(54) Cooking Appliance and Automatic Shut-Off Method for Same

(71) Applicant: Robert Nelson Stanford, Flowery Branch, GA (US)

(72) Inventor: Robert Nelson Stanford, Flowery Branch, GA (US)

(73) Assignee: Calphalon Corporation, Atlanta, GA (US)

(21) Appl. No.: 13/223,206

(22) Filed: Aug. 31, 2011

(51) Int. Cl. A47J 27/62; A23L 1/01; G01N 33/02

(52) U.S. Cl. 426/231; 99/333

(57) ABSTRACT

A cooking appliance has a housing, a cooking medium, a control panel, a heat source for heating the cooking medium, a microprocessor, and a shut-off timer. The timer counts down a specified time when a measured temperature of the cooking medium equals or exceeds a target temperature and shuts off the heat source after the specified time elapses. The microprocessor can pause the shut-off timer when the measured temperature drops below a threshold temperature upon food being placed in contact with the cooking medium. The microprocessor restarts the shut-off timer, if paused, when the measured temperature reaches a predetermined recovery temperature above the threshold temperature. A method of operating such an appliance includes pausing the shut-off timer when the measured temperature drops below the threshold temperature and restarting the shut-off timer when the measured temperature reaches the predetermined recovery temperature.

### BACKGROUND

1. Field of the Disclosure

The present invention is generally directed to cooking appliances, and more particularly to a cooking appliance with an automatic shut-off feature configured to not interrupt a cooking cycle.

2. Description of Related Art

Different types of cooking appliances are known in the art that employ a heated cooking medium that cooks food placed in contact with the cooking medium. Many of these types of appliances have no automatic shut-off feature. Thus, if left unattended, the cooking medium will remain heated, which can render the appliance potentially unsafe. Some of these types of cooking appliances employ an automatic shut-off feature, whereby after a predetermined, set period of time has elapse the heat or power source to the cooking medium is turned off. In some of these types of appliances, the automatic shut-off occurs too quickly, which can result in undercooked food and degrade the performance or perceived performance of the appliance. In some of these types of appliances, the automatic shut-off feature does not take effect until quite a long period of time has elapsed. This can also result in a potentially unsafe appliance, similar to an appliance having no shut-off feature whatsoever.

Automatic shut-off is not required by regulations for cooking appliances or in order to pass Underwriter Laboratory (UL) or other safety compliance testing. However, as noted above, some manufacturers have provided cooking appliances with an automatic shut-off feature. This is sometimes done to put the minds of consumers at ease. This is also sometimes done to provide an additional feature for a particular appliance that may not be offered on other appliances, rendering that particular appliance potentially more attractive to consumers.

One such cooking appliance of the type described above is a deep fryer. Deep fryer's cook food in a vessel that holds a relatively large volume of oil. The oil is heated to relatively high temperatures in order to deep fry the food. Consumers sometimes believe that deep fryers are unsafe, simply because of the presence of the hot oil. Therefore, a number of deep fryer manufacturers offer such appliances with an automatic shut-off feature. These products are configured or preprogrammed to shut off the heating element, heat source, or power to the cooking medium after a predetermined set time has elapsed, following a timed pre-heat cycle. Thus, some manufacturers of this type of appliance have attempted to address this particular safety concern of consumers.

However, having the appliance shut off automatically after a specific elapsed time can cause inconvenience for consumers, particularly those cooking multiple batches of food over a relatively short period of time. Specifically, if the appliance is programmed to shut off after a set elapsed time, it may shut off during a cooking cycle, such as while cooking a second, third, or fourth batch of food. This can result in a lost batch of food, the consumer having to manually restart the appliance, or the food being undercooked and eaten.

### SUMMARY

The disclosed invention is generally directed to a cooking appliance that has an automatic shut-off safety timer. The safety timer will automatically shut-off the heat source to the cooking medium of the appliance after a specified time has elapsed. The shut-off timer is paused, however, during subsequent cooking cycles to avoid the appliance shutting off mid-cycle. The appliance can detect when food is placed in contact with the cooking element or medium and can pause the shut-off timer, allowing the cooking cycle to be completed.

In one example according to the teachings of the present invention, a method of operating a deep fryer includes providing a deep fryer with a reservoir, a heating element, and a controller, and adding oil to the reservoir. The oil is then pre-heated by turning on the heating element. An actual temperature of the oil is then regularly measured. Each time the oil temperature is measured, the measured temperature is transmitted to the controller representative of the actual temperature. The controller then compares the measured temperature to a target temperature. A shut-off timer, which counts down for a specified time, is started when the measured temperature equals or exceeds the target temperature. Food to be cooked is placed in the oil. The shut-off timer is paused if the measured temperature drops to a threshold temperature that is ΔT₁ degrees below the target temperature, ΔT₁ being a predetermined temperature change. The shut-off timer is restarted when the measured temperature recovers ΔT₂ above the threshold temperature, ΔT₂ being a predetermined temperature change. The placing, pausing, and restarting steps can optionally be repeated by a user removing the cooked food from the oil and at some point placing more food to be cooked in the oil. The heating element is automatically shut off when the specified time has elapsed.

In one example, the step of regularly measuring can include positioning a temperature sensor in contact with the oil and the step of transmitting can include repeatedly transmitting a signal from the sensor to the controller representative of the measured temperature.

In one example, the method of operating can include the steps of selecting and setting a pre-set cooking temperature from among a plurality of optionally selectable temperatures.

In one example, the specified time of the starting step can be about 30 minutes.

In one example, the target temperature can be about equal to ΔT₂ plus the threshold temperature of the pausing step.

In one example, ΔT₁ and ΔT₂ can be about equal to one another.

In one example, the target temperature of the pausing step can be a steady state or average of the measured temperatures occurring between the heating step and either the starting step or the placing step.

In one example, ΔT₁ of the pausing step can be about 5 degrees Fahrenheit or more. In one example, ΔT₁ of the pausing step can be about 15 degrees Fahrenheit.

In one example, ΔT₂ can be greater than the threshold temperature but less than the target temperature.

In one example according to the teachings of the present invention, a method of operating a cooking appliance includes the steps of providing an appliance with a cooking medium, a heat source, and a microprocessor and pre-heating the cooking medium. A microprocessor compares a measured temperature of the cooking medium to a target temperature. A shut-off timer, which counts down for a specified time, is started when the measured temperature equals or exceeds the target temperature. Food to be cooked is placed in contact with the cooking medium. The shut-off timer is paused if the measured temperature drops ΔT₁ or more degrees below a threshold temperature, ΔT₁ being a predetermined temperature change. The shut-off timer is restarted when the measured temperature recovers ΔT₂ above the threshold temperature, ΔT₂ being a predetermined temperature change. The steps of placing, pausing, and restarting can be optionally repeated. The heat source is automatically shut off when the specified time has elapsed.

In one example, the step of providing can include providing a deep fryer with a reservoir and the cooking medium can be cooking oil retained in the reservoir.

In one example, the method of operating can further include the steps of selecting and setting the target temperature as a pre-set cooking temperature from among a plurality of optionally selectable temperatures.

In one example, the specified time of the starting step can be about 30 minutes. Also, ΔT₁ of the pausing step can be 5 degrees Fahrenheit or more.

In one example, the target temperature can be a user selected pre-set cooking temperature or an average of the measured temperatures occurring between the pre-heating step and either the pausing step, the placing step, or the starting step.

In one example, the method of operating can further include two or more occurrences of the placing step.

In one example, the restarting step can further include continuing the count down of the specified duration from where it stopped during the previous pausing step.

In one example, the restarting step can further include resetting the shut-off timer to count down from the beginning of the specified time or from the beginning of a new predetermined restart time different from the specified time.

In one example, the method of operating can further include the steps of selecting and setting the ΔT₁ degrees from among a plurality of selectable ΔT₁ options.

In one example according to the teachings of the present invention, a cooking appliance has a housing, a cooking medium defined at least in part by the housing, a control panel with controls operable by a user to operate the deep fryer, and a heat source for heating the cooking medium. A microprocessor is in electronic communication with the heat source and the controls. A shut-off timer that is controlled by the microprocessor starts counting down a specified time when a measured temperature of the cooking medium equals or exceeds a target temperature and shuts off the heat source after counting down for the specified time. The microprocessor can pause the shut-off timer, i.e., temporarily stop the count down, when the measured temperature drops below a threshold temperature upon food being placed in contact with the cooking medium. The microprocessor restarts the shut-off timer, if paused, when the measured temperature recovers by a predetermined recovery temperature above the threshold temperature.

In one example, the microprocessor can be operable by a user to select the target temperature as a pre-set cooking temperature and/or can be operable by the user to pre-set the threshold temperature.

In one example, the cooking appliance can further include a temperature sensor in communication with the microprocessor and with the cooking medium.

In one example, the controls can include a temperature control configured to allow a user to select and set a pre-set cooking temperature. The target temperature can be equal to the pre-set cooking temperature or can be an average temperature of the cooking medium prior to the shut-off timer being paused.

In one example, the controls can include a timer control configured to allow a user to select the specified time for the shut-off timer.

### BRIEF DESCRIPTION OF THE DRAWINGS

Objects, features, and advantages of the present invention will become apparent upon reading the following description in conjunction with the drawing figures, in which:

FIG. 1 shows one example of a cooking appliance in the form of the deep fryer constructed in accordance with the teachings of the present invention.

FIG. 2 shows a control panel of the cooking appliance shown in FIG. 1.

FIG. 3 shows a side view of the cooking appliance shown in FIG. 1 with a side wall cut away and depicting a simplified schematic of the control unit of the appliance.

FIG. 4 shows a flowchart of one example of a method of operating a cooking appliance in accordance with the teachings of the present invention.

FIG. 5 shows a plot of temperature versus time representative of the method shown in FIG. 4 and while cooking French fries in a deep fryer.

FIG. 6 shows a plot of temperature versus time representative of the method shown in FIG. 4 and while cooking chicken tenders in a deep fryer.

### DETAILED DESCRIPTION OF THE DISCLOSURE

The present invention is directed to cooking appliances and automatic shut-off methods for such appliances. The disclosed appliance and method of operation examples solve or improve upon one or more of the above-noted and/or other problems and disadvantages with prior known cooking appliances. In one example, a disclosed cooking appliance has a microprocessor or controller configured to automatically shut off the cooking medium after a specified time has elapsed. However, the specified time, i.e. the shut-off countdown, can be paused or delayed while food is being cooked. In one example, a disclosed cooking appliance can detect when food is being cooked by sensing a temperature drop of the cooking medium being measured. Thus, a disclosed cooking appliance can allow a user to cook multiple batches of food in a relatively short period of time without the automatic shut-off feature taking effect and shutting down the appliance during a cooking cycle.

In one example, a disclosed method of operating a cooking appliance includes starting a shut-off timer when a cooking medium of the appliance reaches a target temperature. The automatic shut-off timer can be paused or delayed while food is being cooked by the appliance. In one example, a disclosed method of operating a cooking appliance includes restarting the shut-off timer, after having been paused; when it is determined that food is no longer being cooked by the appliance. In one example, a disclosed method of operating a cooking appliance pauses and restarts the shut-off timer according to variations in the measured temperature of the cooking medium. These and other objects, features, and advantages of the present invention will become apparent to those having ordinary skill in the art upon reading this disclosure.

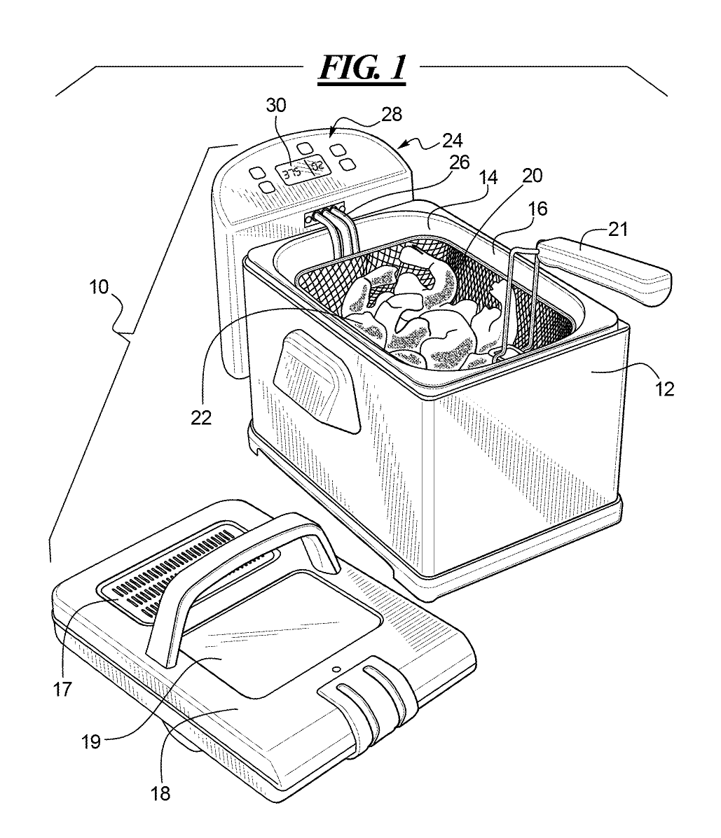

Turning now to the drawings, FIGS. 1-3 illustrate one example of a cooking appliance constructed in accordance with the teachings of the present invention. In this disclosed example, the cooking appliance is a deep fryer 10 depicted in FIG. 1 and generally has a base unit or housing 12, which defines a cooking reservoir 14 therein, as is known in the art. The housing 12 has a top opening 16 into the reservoir 14 that can be selectively covered by a lid or cover 18. In one example, the reservoir 14 can include a durable, washable, and removable receptacle, such as a glass bowl, metal bowl, enamel coated steel bowl or the like, that can be removed, easily cleaned, and replaced, as needed. The lid 18 can include a glass window 19 so that the user can see the interior of the deep fryer during use. The lid 18 can alternatively be made entirely of glass, if desired. The lid can also include one or more vents 17 and/or filters, and the like. The vents 17 can be provided to allow gases to escape the deep fryer during use. The filters can be provided to capture or prevent oil and other contaminants or residue from escaping during use of the deep fryer.

A basket 20 can be removably placed in the reservoir 14 and can be filled with food 22 to be cooked. The basket 20 can include a handle 21 that protrudes from the reservoir 14, making it easy for a user to remove and replace the basket while leaving the cooking oil behind in the reservoir 14. Oil 23 (see FIG. 3) is added to the reservoir 14 in order to cook the food 22. In this type of cooking appliance, the oil 23 acts as the cooking medium. The food 22 is placed in the reservoir in contact with the oil, i.e., the cooking medium, when the oil is adequately heated. The oil 23 can be any suitable cooking oil and is not shown or further described in any further detail herein.

In this example, the deep fryer 10 has a separate control unit 24 with the heating element 26 projecting therefrom. The heating element 26 in this example is an immersion-type rod assembly that projects outward and downward from the control unit 24. The control unit 24 can be connected via a side wall mount 27 to a wall of the housing 12 with the heating element 26 depending into the reservoir 14. The mount 27 can include a safety switch (not shown) that closes only when the control unit is completely attached and connected to the housing. The heating element 26 will thus be immersed in the oil 23 within the reservoir 14 to heat the oil. Though not shown herein, the control unit 24 can include an electrical cord for connecting the deep fryer, and particularly the control unit, to an external, household AC power source or some other suitable power source. The immersion rod of the heating element 26 can include a U-shaped continuous tube, rod, or wire protruding from the control unit 24. As described below, a temperature sensor 50 can be positioned between the legs of the U-shaped rod, wire, or tube of the immersion rod structure.

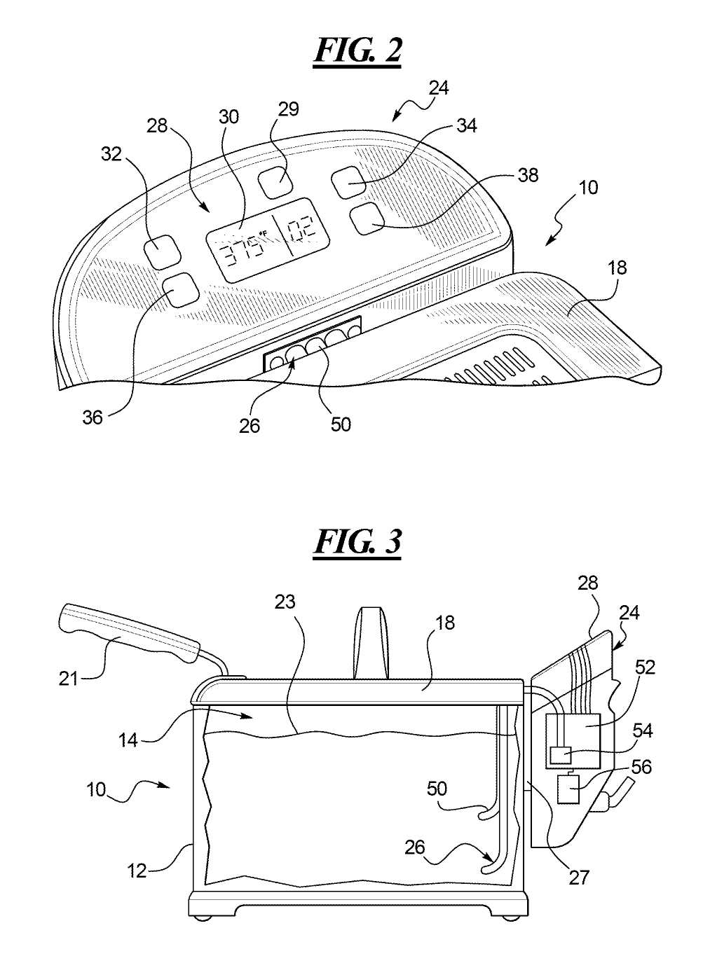

In this example, a user can operate the deep fryer 10 by actuating a plurality of operator controls provided on a control panel 28. The control panel 28 of the deep fryer 10 is positioned on top of the control unit 24 in this example and is illustrated in FIG. 2. In one example, the control panel 28 can include a power or ON/OFF button 29 and a display 30, such as an LCD display, an LED backlit display, a touch screen, or the like. In this example, the deep fryer 10 is turned on simply by plugging in the power cord. The button 29 can be used to start a timer, cooking program, or the like, or to enter or lock in other user operable selections and settings. The user can then select a desired cooking time and set a cooking timer by manipulating a timer select button 34 on the control panel 28 until the desired time appears on the display 30. The user can also select a desired or pre-set cooking temperature by manipulating a temperature select button 32 on the control panel 28 until the desired cooking temperature appears on the display 30. The user can pre-heat the oil retained in the reservoir 14 by pressing or manipulating a temp START/CANCEL button 36 on the control panel 28. The heating element 26 will then be heated, which in turn will begin to heat the oil 23. When the oil 23 is ready, i.e., when it has reached the pre-set temperature, the user can also start the cooking timer by manipulating or pressing a time START/CANCEL button 38 on the control panel 28. The user will be notified via an audible or other suitable signal that the selected cooking time has elapsed.

The disclosed deep fryer 10 is only one of numerous types of cooking appliances that may benefit from using the inventions disclosed herein. Other cooking appliances have cooking mediums, controls, and heating elements that may be programmed and controlled to function in a similar matter. Waffle irons, toasters, toaster ovens, bread making machines, stove burners, ovens, and the like may utilize and benefit from the methods of operating a cooking appliance as disclosed herein. For example, a waffle iron has two direct contact heating plates that act as the cooking medium and an electric heating element to heat the plates. Ovens and grills use a confined space, grates, and radiant heat within the space as the cooking medium and use heat emanating from a heating element (gas burner, charcoal, heated coils, etc.) to heat the medium. Gas stove tops utilize a flame as the heating element and a grate and a cookware product (pot, pan, skillet, etc.) as the cooking medium. Electric stove tops similarly use a heated coil as the heating element a cookware product as the direct contact cooking medium. The heating elements and cooking mediums on these types of cooking appliances can be controlled, operated, and shut off in accordance with the teachings of the present invention.

Also, the operator controls, including the control unit 28, the various buttons 29 and 32-38, and the display 30 that are discussed above, can also vary, as described in further detail below. The cooking medium and heating element can also vary, as noted above, from the immersion rod, oil, and reservoir of the disclosed deep fryer 10. Many different types of cooking appliances could benefit from and utilize the methods of operating cooking appliances as disclosed herein.

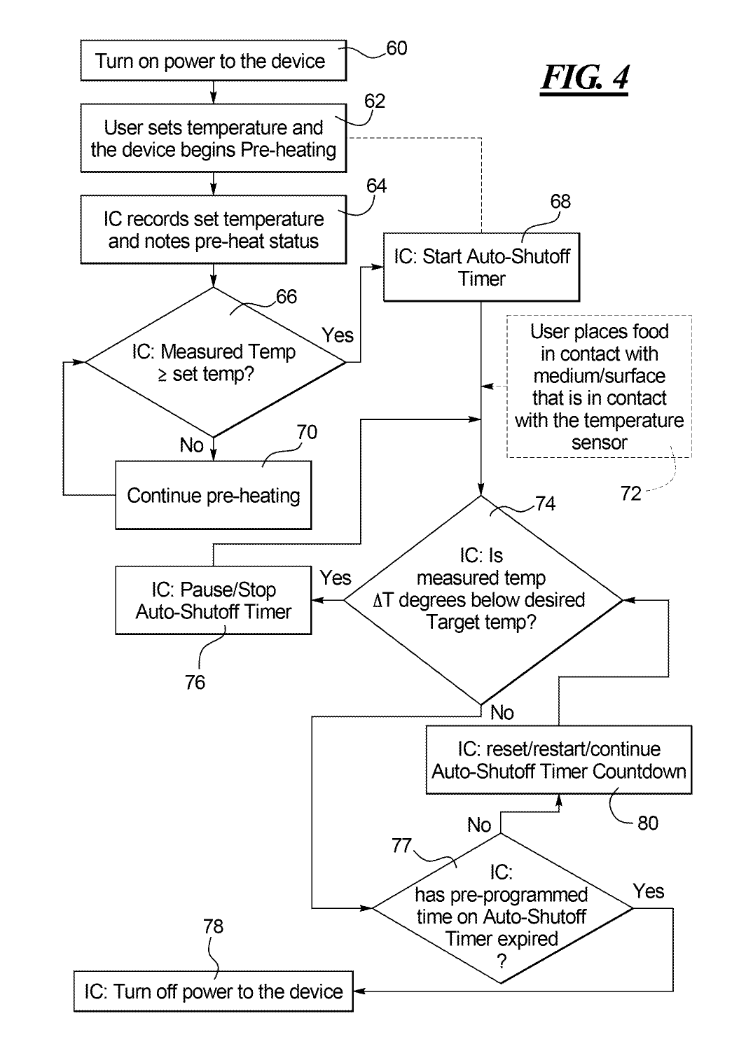

FIG. 3 illustrates one example in simplified schematic form of the relationship and connection between the various functional components of the disclosed deep fryer 10 in this example. As mentioned above, the temperature sensor 50 is positioned within or in close proximity to the reservoir 14 so as to measure an actual temperature of the cooking medium, i.e., the cooking oil, of the deep fryer 10. The deep fryer has a microprocessor 52, master controller, micro-controller, or the like, which in this example is housed in the control unit 24. The temperature sensor 50 is electrically connected to the microprocessor 52. Similarly, the display 30 and each of the buttons 32-38 are connected to the microprocessor 52. The heating element 26 is also connected to the microprocessor 52 and can be controlled thereby. In this example, the microprocessor 52 has an integrated circuit 54, which can be configured and programmed to control the various components and functions of the deep fryer 10. A shut-off timer 56 is also provided within the control unit 24 and can be a part of the integrated circuit 54, the microprocessor 52, or as a separate component electrically connected to the integrated circuit. Details of the microprocessor function and the method of operating the deep fryer 10 are discussed in greater detail with reference to FIG. 4.

However, in general, the microprocessor 52 can be programmed, and the control panel 28 can be configured, to allow a user to control various other functions and parameters of the cooking appliance, i.e., the deep fryer 10 in this example, and to monitor various other functions and parameters as well. For example, the temperature sensor 50 and microprocessor 52 can be configured to measure and monitor an actual temperature of the oil in the reservoir 14. The microprocessor can also be configured to store the measured temperature data and can be configured to continuously, periodically, intermittently, regularly, frequently, or otherwise receive and store the measured temperature data of the oil and to calculate other parameters and variables based on the measured temperature.

FIG. 4 illustrates a flow chart of one example of a method for operating a cooking appliance, and in particular, with reference to the deep fryer 10 disclosed and described above and shown in FIGS. 1-3. Some initial, conventional steps of operating the deep fryer 10 are not represented in FIG. 4. For example, one must first procure or provide a cooking appliance, such as the deep fryer 10, and then operate the appliance. For the deep fryer 10, oil must first be added to the reservoir 14. For other types of cooking appliances, this step is not necessary. Power to the cooking appliance must be turned on or connected or energy must be provided to heat, light, or energize the heating element of the cooking appliance. This initial step is represented at block 60 in FIG. 4.

Once the appliance is powered up or supplied with energy, the user can then select various operational parameters of the cooking appliance, such as the above-described cooking cycle timer (buttons 34, 38) and pre-set cooking temperature (buttons 32 and 36). In another example, the user may be able to select from a number of optional food cooking programs specifically programmed for particular types of food. The programs can automatically set the various parameters of the appliance, if desired. In any event, the desired cooking temperature for the cooking medium is defined herein as the pre-set cooking temperature for the appliance. This step is represented at block 62. The user can then start the appliance to energize or heat the heating element. This in turn will pre-heat the cooking medium of the appliance. In this example, the deep fryer 10 is powered up and turned on by plugging in the cord, selecting the parameters, and pressing the button 29. Whether manually, automatically, or by program selection, the user selects the desired cooking temperature and cooking time and sets the timer and the pre-set cooking temperature accordingly using the control panel 28, display 30, and buttons 32-38. The deep fryer can then be turned on by pressing the button 29 to heat the heating element 26 and pre-heat the oil 23. This step is also represented at block 62.

The microprocessor 52 can store the pre-set cooking temperature selected by the user. This step is represented at block 64. The temperature of the cooking medium can be regularly measured and transmitted to the processor or controller at regular intervals. In this example, the temperature sensor 50 can transmit the measured temperature, representative of the real time actual temperature of the oil 23 in the reservoir 14, to the microprocessor 52. This transmitting step can also be done continuously or at any desired interval, i.e., regularly, periodically, intermittently, frequently, repeatedly, or the like. Thus, the temperature sensor 50 and microprocessor 52 can be configured to measure and monitor the actual temperature of the oil in the reservoir 14 during and after the pre-heat step. This step is also represented at block 64 in FIG. 4.

The measured temperature of the cooking medium can be compared to a target temperature periodically. In this example, the measured temperature of the oil 23 can be initially compared to the pre-set temperature by the microprocessor 52. Thus, the target temperature can equal the pre-set cooking temperature. This step can be performed to determine when the cooking medium, such as the oil in this example, has reached the pre-set temperature and is ready for cooking food. This initial comparison step is represented at block 66 in FIG. 4. The microprocessor can be programmed to perform this initial comparison step continuously or at any suitable interval, as desired.

It is possible that the actual temperature of the medium does not achieve the pre-set cooking temperature, or only after the initial pre-heat cycle. The microprocessor 52 can be programmed or configured to determine when the measured temperature achieves some reference or target temperature that is incrementally below the pre-set cooking temperature to avoid this problem. Alternatively, the target temperature can be a steady state or average temperature calculated over a period of time. In one example, the microprocessor can determine an average temperature of the cooking medium, after having completed a pre-heat or warm-up phase. Once the cooking medium temperature has leveled off to a steady state, the steady state temperature can be the target temperature for the comparison, if desired. As used herein, the target temperature is optionally interchangeable with such incrementally lower reference temperature, the pre-set cooking temperature, or the steady state stored average temperature of the cooking medium.

In the disclosed example, the deep fryer 10 is configured having an automatic shut-off feature controlled by the shut-off timer 56 noted above. In one example, the shut-off timer 56 can be programmed to start after a predetermined pre-heat time for the cooking medium. In another example, represented in FIG. 4 as a dashed line between blocks 68 and 62, the shut-off timer can be configured to start when the pre-heat cycle starts, the shut-off time accounting for the pre-heat time duration. Alternatively, the shut-off timer 56 can be configured to start only after the regular temperature comparison indicates that the cooking medium is heated to the pre-set cooking temperature or some other target temperature parameter. Thus, the shut-off timer 56 in this example can be started when the measured temperature of the oil equals or exceeds the target temperature or pre-set cooking temperature of the oil 23. This step is represented at block 68. When turned on, the shut-off timer 56 begins a count down that is set for a specified time or shut-off time. If the measured temperature of the oil remains less than the target temperature, the step of pre-heating the cooking medium will continue as represented at block 70. Also, the shut-off timer 56 will not yet be turned on.

In one example, the specified time for the shut-off timer 56 can be about 30 min. The countdown time or specified time can vary from the disclosed 30 min. example, if desired. In another example, the shut-off timer 56 can be user adjustable within a predetermined specified time range. Thus, in such an alternative example the specified time can be adjusted by the user according to the needs of a particular cooking appliance, type of food to be cooked, number of expected cooking cycles, or the like. The shut-off timer 56 can also be programmed to have a delayed start for a predetermined time after the measured temperature first achieves the target temperature. Alternatively, the specified time can account for an initial cooking cycle or two without risk of the cooking appliance being automatically shut off. If food is never placed in or on the cooking medium once it is pre-heated and ready for cooking, the specified time of the shut-off timer will elapse and then shut off the cooking appliance. The shut-off timer function is provided as a safety feature to prevent the cooking appliance from being accidentally or inadvertently left on indefinitely.

More typically, the user will place food to be cooked in contact with the cooking medium. In the disclosed example, the user can place the food 22 into the basket 20, which can then be placed into the reservoir 14 of the deep fryer 10. In other examples, the user can place food in contact with or into the appropriate cooking medium after the cooking medium has been pre-heated. This step is represented at block 72. In accordance with the teachings of the present invention, once food is placed in contact with the cooking medium, such as the oil 23 in the deep fryer 10, the shut-off timer 56 can be paused to prevent premature termination of a cooking cycle in the appliance.

In the present example, by placing food 22 into the oil within the reservoir 14, the actual temperature of the oil will suddenly drop or decrease rapidly to a temperature measurably below the pre-set temperature. The automatic shut-off method according to the teachings of the present invention utilizes this sudden or rapid temperature drop as an indication or signal that food is currently being cooked by the appliance and that the count down of the shut-off timer 56 should be paused or stopped. In one example, the measured temperature of the oil can be compared to a desired threshold temperature to make this determination. This step is represented at block 74. The threshold temperature can be a temperature that is a predetermined amount below the target temperature. This threshold temperature can vary, depending on the type of food being cooked and the target temperature being utilized.

The threshold temperature can be set at ΔT₁ degrees less than the target temperature, where ΔT₁ is a predetermined temperature change or drop. If the microprocessor determines that the measured temperature of the oil is equal to or less than the threshold temperature, i.e., is ΔT₁ degrees less than the target temperature, the microprocessor can pause the shut-off timer 56. This will temporarily stop the specified time countdown. This step is represented at block 76 in FIG. 4. However, the measured temperature comparison will be continued, as at block 74. If the microprocessor determines that the measured temperature is not below the threshold temperature, the shut-off timer 56 is continued. The microprocessor 52 will also determine whether the specified time of the shut-off timer 56 has expired or elapsed. This step is represented at block 77. If the specified time has elapsed, power to the cooking medium is turned off. In the disclosed example, this would mean that the immersion rod heating element 26 would be turned off. This step is represented at block 78. If the specified time of the shut-off timer 56 has not elapsed, the microprocessor would then allow the shut-off timer 56 to continue counting down, if not paused, or would restart the shut-off timer if it had been paused. This step is represented at block 80 in FIG. 4.

The microprocessor will continue to repeat the temperature comparison of block 74. If a food batch was previously cooked in the oil and then removed, the measured temperature of the oil will recover and again approach the target temperature. This will cause the microprocessor to repeat or loop between blocks 74, 76, 77, and 80 until the specified time counts down. The user can also optionally repeat step 72, i.e., placing food into contact with the cooking medium one or more times. Either a user places another batch of food into the oil as at block 72, or the specified time of the shut-off timer 56 eventually will elapse, as at blocks 77 and 78. If another food batch is placed in the oil, the temperature of the oil will again drop suddenly. The temperature comparison at block 74 will again result in the shut-off timer 56 being paused as the food is cooked in the oil. The automatic shut-off method disclosed and described herein allows a user to cook multiple batches of food because the shut-off timer 56 will be in sleep mode, resulting in the specified time countdown being paused.

The timer countdown will generally only continue when food is not present in the cooking oil. However, it is possible that even if food is cooking in or on the cooking medium, the measured temperature will rise high enough that the timer will start up again. The integrated circuit 54 can be programmed to keep the shut-off timer 56 in the paused state, either for a period of time after the measured temperature again rises above the threshold temperature, or until a certain recovery temperature of the oil or cooking medium is reached. This may help in avoiding the shut-off timer from prematurely starting up again during a cooking cycle, which could otherwise risk shutting down the heating element mid-cycle.

For example, the shut-off timer 56 can stay in sleep mode until the measured temperature recovers ΔT₂ above the threshold temperature, where ΔT₂ is again a predetermined temperature change. In one example, ΔT₂ can be a small incremental temperature above the threshold temperature. In another example, ΔT₂ can result in a recovery temperature somewhere between the target temperature and the threshold temperature. In yet another example, ΔT₂ can be about equal to ΔT₁, which would result in the recovery temperature being essentially equal to the target temperature. In any case, as long as the recovery temperature is above the threshold temperature, the shut-off timer 56 would not immediately start back up as soon as the measured temperature was again to exceed the threshold temperature.

A number of parameters in the above described method can vary within the spirit and scope of the present invention. For example, the temperature drop ΔT₁ can vary. In one example, the shut-off timer 56 can be paused only if ΔT₁ is 15° or more, i.e., the threshold temperature is 15° less than the pre-set temperature. In other examples, the shut-off timer 56 can be paused if the threshold temperature is 10°, or even only 5°, less than the pre-set temperature. Similarly, the recovery temperature parameter ΔT₂ can also vary, as noted above. These temperature variables can also vary, depending on the type of food to be cooked. Also, the target temperature definition can vary from the options defined herein.

Different types of food may affect the temperature of the cooking medium in different ways. Large volume frozen foods, such as French fries or chicken tenders, may cause the cooking medium temperature to drop more quickly and by a greater margin than lesser volume, thawed foods. See two examples discussed briefly below. Thus, cooking appliances can be configured having different threshold temperatures or an alternate ΔT₁ temperature differential that causes the shut-off timer to delay or pause. Some foods, such as donuts or thin fish filets, may only result in a 4° or 5° drop in oil temperature when placed in the reservoir to be cooked. Thus, ΔT₁ would necessarily be only a few degrees Fahrenheit.

In one example, the control unit 28, including the microprocessor 52, integrated circuit 54, and the like, can be configured to allow a user to adjust or select the ΔT₁ temperature difference or the shut-off threshold temperature. The selected temperature differential or threshold temperature will depend on the type and necessary cooking temperature of the food to be cooked. In another example, the control unit 28 can be configured to contain a number of different user selectable cooking programs that are each food specific. Thus, a user, when turning on the cooking appliance and selecting or setting the cooking timer and cooking temperature settings, could instead or in addition also select a specific cooking program to accommodate a specific food type to be cooked. As noted above, the program could automatically set one or more of the cycle parameters, such as the pre-set cooking temperature, the cooking time, the threshold temperature, the specified time for the shut-off timer, ΔT₁, ΔT₂, how the target temperature is determined, and/or the like.

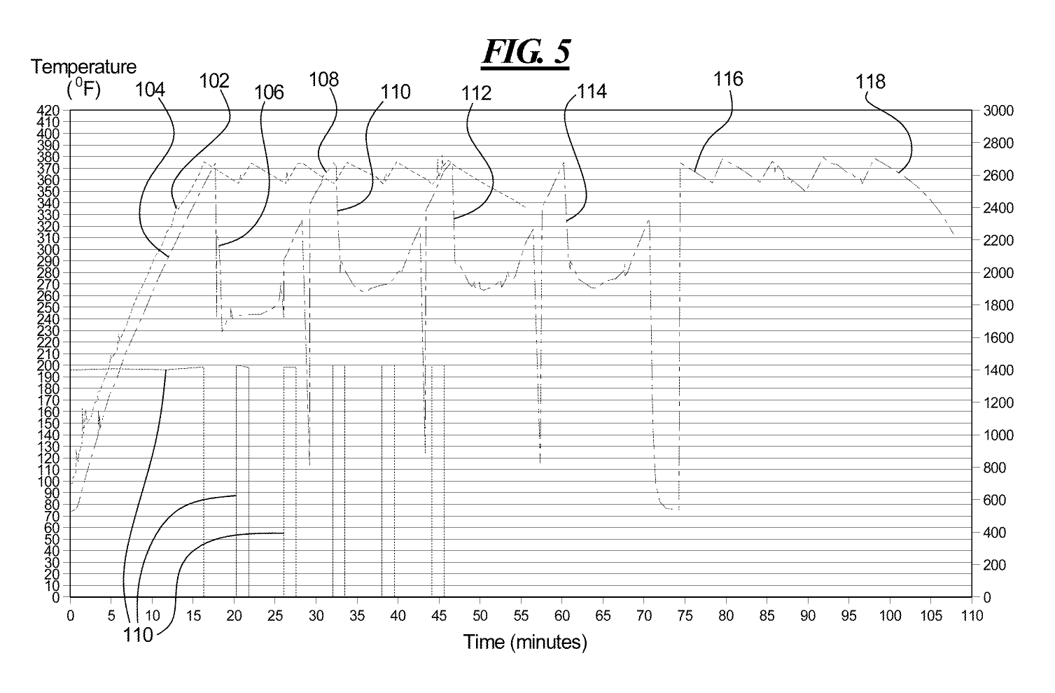

FIG. 5 shows the results of one test example using a deep fryer as the cooking appliance and French fries as the food to be cooked. A plot of temperature vs. time for two deep fryers is shown in the figure. A first line 100 is also shown and represents when power was either on or off to the heating element. The heating element was powered intermittently in this test deep fryer to maintain a relatively constant steady state once reached. A second line 102 is representative of temperature vs. time for a reference deep fryer in which no food was cooked. As can be seen, power was applied to pre-heat the oil, i.e., the cooking medium, to a pre-set temperature of 375° F. Once the measured temperature reached the pre-set temperature, the shut-off timer was started and counted down 30 minutes, i.e., the specified time in this example. The heating element was also intermittently energized to maintain the oil temperature between the threshold temperature and the pre-set temperature while the specified time had not yet elapsed. The pre-heat time was about 15 minutes for the reference deep fryer according to line 102. After the specified time had elapsed, the shut-off timer signaled the microprocessor to shut off the heating element, as an indication that the user either was not going to cook any food or not going to cook any more food. The oil then slowly began to cool down, as the end of the line 102 indicates.

Line 104 represents a test deep fryer used to cook French fries. As can be seen, the pre-heat time and temperature curves are essentially identical. The shut-off timer started at the same time as the reference deep fryer of line 102. A first food batch was placed in the oil, as depicted at portion 106 of the curve. The temperature spiked well below the pre-set temperature when the food was placed in the oil and then slowly recovered as the French fries cooked. When the first batch was done, the cooked French fries were removed. During or after this time, the oil temperature rose to about the pre-set temperature. Depending on the values of ΔT₁ and ΔT₂, the shut-off timer would restart or reset, such as at portion 108 of the line 104. Another food batch to be cooked was then placed in the oil as shown at portion 110 of the line 104 and again the oil temperature dropped or spiked well below the pre-set temperature and threshold temperature. This was repeated for two more food batches as represented at portions 112 and 114 of the line 104, after which no more food was cooked. The line portion 116 represents the oil temperature at this stage. The shut-off time counted down intermittently between food batches. Then the remainder of the specified time of 30 minutes counted down after the last batch. The heating element was then shut off as represented at the end 118 of the line 104.

In this example, the Chart 1 below shows significant data and test parameters for the test and reference deep fryers represented in FIG. 5. The shut-off timer on the reference deep fryer continued to run for about 30 minutes after starting because no food was cooked and the measured temperature of the oil stayed above the threshold temperature. The target temperature was below the 375° F. pre-set temperature. The shut-off timer for the test deep fryer paused for a total of about 49 minutes during the multiple cooking cycles. The target temperature used by the microprocessor of the test deep fryer was the average measure temperature of the oil, after preheat, and not the pre-set 375° F. As noted above, the cooking medium will not always achieve and maintain the desired pre-set temperature because it will fluctuate during cooking cycles. In this example, ΔT₁ was about 15° F. below the average oil temperature.

CHART 1

| | | | Thresh- | | | | |
|---|---|---|---|---|---|---|---|
| | | | old | Total | | Timer | Timer |
| | | | Temp | ON | Preheat | ON | OFF |
| | Preheat | Average | (avg- | time | time | time | time |
| | Temp | Temp | 15°) | (mins) | (mins) | (mins) | (mins) |
| Ref | 375° F. | 368° F. | 353° F. | 45.68 | 16.32 | 29.37 | — |
| Deep | | | | min | min | min | |
| Fryer | | | | | | | |
| Test | 375° F. | 367° F. | 352° F. | 98.30 | 17.87 | 31.78 | 48.65 |
| Deep | | | | min | min | min | min |
| Fryer | | | | | | | |

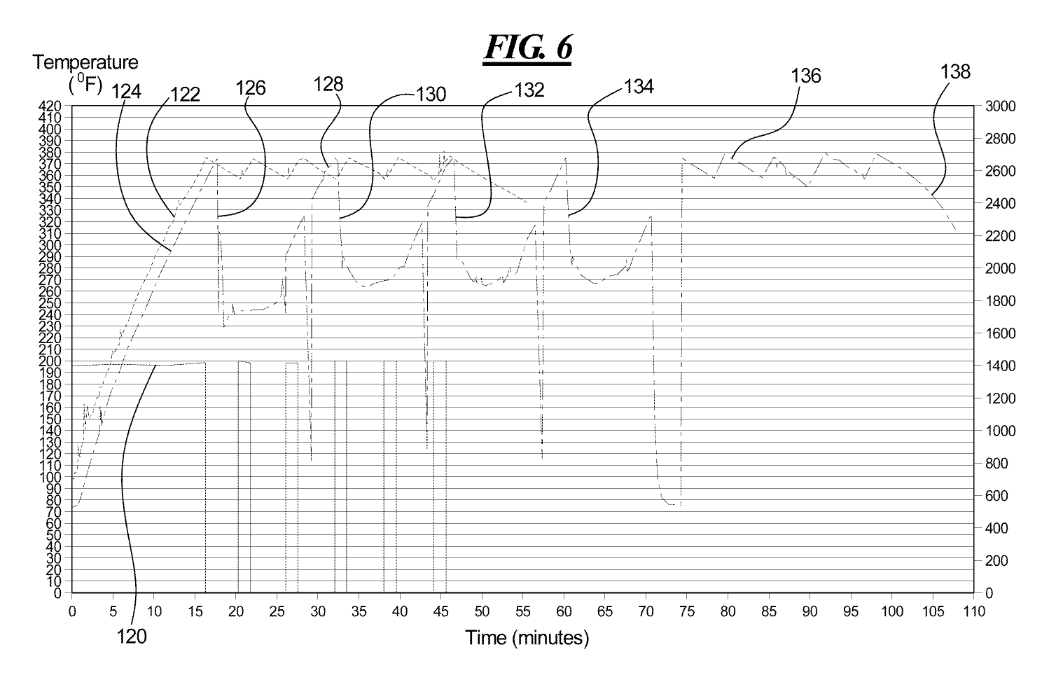

FIG. 6 shows the results of another test example using a deep fryer as the cooking appliance and chicken tenders as the food to be cooked. A plot of temperature vs. time for two deep fryers is again shown in the figure. A first line 120 is also shown and represents when power is either on or off to the heating element, same as for the above described example in FIG. 5. A second line 122 is representative of temperature vs. time for the reference deep fryer in which no food is cooked. This line is essentially the same as the line 102 for the first example above, except that the pre-set temperature was 350° F.

A third line 124 represents a test deep fryer used to cook the chicken tenders. As can be seen, the pre-heat time and temperature curves are again essentially identical. The shut-off timer started at the same time as the reference deep fryer of line 122. A first chicken tender batch was placed in the oil, as depicted at portion 126 of the curve. The temperature again spiked well below the pre-set temperature and then slowly recovered as the chicken tenders cooked. When the first batch was done, the cooked chicken tenders were removed. During or after this time, the oil temperature rose to about the pre-set temperature. Again, depending on the values of ΔT₁ and ΔT₂, the shut-off timer will restart or reset at portion 128 of the line 124. Three more food batches were cooked as represented at portions 130, 132, and 134 of the line 124, after which no more food was cooked. The line portion 136 represents the oil temperature at this stage. The shut-off time counted down intermittently between the four food cycles, and then the remainder of the specified time of 30 minutes elapsed after the last batch. Then the heating element was shut off as represented at the end 138 of the line 124.

In this example, the Chart 2 below shows significant data and test parameters for the test and reference deep fryers represented in FIG. 6. The shut-off timer on the reference deep fryer continued to run for about 30 minutes after starting because no food was cooked. The shut-off timer for the test deep fryer paused for a total of about 20 minutes during the multiple cooking cycles. The target temperature used by the microprocessor of the test deep fryer was again the average measured temperature of the oil, after preheat, and not the pre-set 350° F. In this example, ΔT₁ was again about 15° F. below the average oil temperature.

CHART 2

| | | | Thresh- | | | | |
|---|---|---|---|---|---|---|---|
| | | | old | Total | | Timer | Timer |
| | | | Temp | ON | Preheat | ON | OFF |
| | Preheat | Average | (avg- | time | time | time | time |
| | Temp | Temp | 15°) | (mins) | (mins) | (mins) | (mins) |
| Ref | 350° F. | 346° F. | 331° F. | 45.62 | 16.15 | 29.47 | — |
| Deep | | | | min | min | min | |
| Fryer | | | | | | | |
| Test | 350° F. | 347° F. | 332° F. | 67.63 | 14.82 | 33.23 | 19.88 |
| Deep | | | | min | min | min | min |
| Fryer | | | | | | | |

Although certain cooking appliances and methods of automatically shutting off cooking appliances have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A method of operating a deep fryer, the method comprising the steps of:
providing a deep fryer with a reservoir, a heating element, and a controller;
adding oil to the reservoir;
pre-heating the oil by turning on the heating element;
regularly measuring an actual temperature of the oil;
transmitting a measured temperature of the oil to the controller representative of the actual temperature;
comparing via the controller the measured temperature to a target temperature;
starting a shut-off timer, which counts down for a specified time, when the measured temperature equals or exceeds the target temperature;
placing food to be cooked in the oil;
pausing the shut-off timer if the measured temperature drops to a threshold temperature that is ΔT₁ degrees below the target temperature, ΔT₁ being a predetermined temperature change;
restarting the shut-off timer when the measured temperature recovers ΔT₂ above the threshold temperature, ΔT₂ being a predetermined temperature change;
optionally repeating the placing, pausing, and restarting steps; and
automatically shutting off the heating element when the specified time has elapsed.

2. The method of operating a deep fryer according to claim 1, wherein the step of regularly measuring includes positioning a temperature sensor in contact with the oil and wherein the step of transmitting includes repeatedly transmitting a signal from the sensor to the controller representative of the measured temperature.

3. The method of operating a deep fryer according to claim 1, further comprising the steps of selecting and setting the target temperature as a pre-set cooking temperature from among a plurality of optionally selectable temperatures.

4. The method of operating a deep fryer according to claim 1, wherein the specified time of the starting step is about 30 minutes.

5. The method of operating a deep fryer according to claim 1, wherein ΔT₂ plus the threshold temperature of the pausing step is equal to the target temperature.

6. The method of operating a deep fryer according to claim 1, wherein ΔT₁ and ΔT₂ are about equal to one another.

7. The method of operating a deep fryer according to claim 1, wherein the target temperature of the pausing step is an average of the measured temperatures occurring between the pre-heating step and either the starting step or the placing step.

8. The method of operating a deep fryer according to claim 1, wherein ΔT₁ of the pausing step is about 5 degrees Fahrenheit or more.

9. The method of operating a deep fryer according to claim 1, wherein ΔT₁ of the pausing step is about 15 degrees Fahrenheit.

10. The method of operating a deep fryer according to claim 1, wherein ΔT₂ is greater than the threshold temperature but less than the target temperature.

11. The method of operating a deep fryer according to claim 1, wherein the target temperature of the pausing step is equal to a pre-set cooking temperature selected for the food to be cooked.

12. A method of operating a cooking appliance, the method comprising the steps of:
providing an appliance with a cooking medium, a heat source, and a microprocessor;
pre-heating the cooking medium;
comparing via the microprocessor a measured temperature of the cooking medium to a target temperature;
starting a shut-off timer, which counts down for a specified time, when the measured temperature equals or exceeds the target temperature;
placing food to be cooked in contact with the cooking medium;
pausing the shut-off timer if the measured temperature drops ΔT₁ or more degrees below a threshold temperature, ΔT₁ being a predetermined temperature change;
restarting the shut-off timer when the measured temperature recovers ΔT₂ above the threshold temperature, ΔT₂ being a predetermined temperature change;
optionally repeating the placing, pausing, and restarting steps; and
automatically shutting off the heat source when the specified time has elapsed.

13. The method of operating a cooking appliance according to claim 12, wherein the step of providing includes providing a deep fryer with a reservoir, and wherein the cooking medium is cooking oil retained in the reservoir.

14. The method of operating a cooking appliance according to claim 12, further comprising the steps of selecting and setting the target temperature as a pre-set cooking temperature from among a plurality of optionally selectable temperatures.

15. The method of operating a cooking appliance according to claim 12, wherein the specified time of the starting step is about 30 minutes, and wherein ΔT₁ of the pausing step is 5 degrees Fahrenheit or more.

16. The method of operating a cooking appliance according to claim 12, wherein the target temperature is a user selected pre-set cooking temperature or an average of the measured temperatures occurring between the pre-heating step and either the pausing step, the placing step, or the starting step.

17. The method of operating a cooking appliance according to claim 12, further comprising two or more occurrences of the placing step.

18. The method of operating a cooking appliance according to claim 12, wherein the restarting step further includes continuing the count down of the specified duration from where it stopped during the pausing step.

19. The method of operating a cooking appliance according to claim 12, wherein the restarting step further includes resetting the shut-off timer to count down from the beginning of the specified time or from the beginning of a new predetermined restart time different from the specified time.

20. The method of operating a cooking appliance according to claim 12, further comprising the steps of selecting and setting the ΔT₁ degrees from among a plurality of selectable temperature change options.

21. A cooking appliance comprising:
a housing;
a cooking medium defined at least in part by the housing;
a control panel with controls operable by a user to operate the cooking appliance;
a heat source for heating the cooking medium;
a microprocessor in electronic communication with the heat source and the controls; and
a shut-off timer controlled by the microprocessor that starts counting down a specified time when a measured temperature of the cooking medium equals or exceeds a target temperature and that shuts off the heat source after counting down for the specified time,
wherein the microprocessor can pause the shut-off timer count down when the measured temperature drops below a threshold temperature upon food being placed in contact with the cooking medium, and wherein the microprocessor restarts the shut-off timer, if paused, when the measured temperature recovers by a predetermined recovery temperature above the threshold temperature.

22. The cooking appliance according to claim 21, wherein the microprocessor is operable by a user to pre-set the target temperature to a desired cooking temperature and/or to pre-set the threshold temperature.

23. The cooking appliance according to claim 21, further comprising a temperature sensor in communication with the microprocessor and with the cooking medium.

24. The cooking appliance according to claim 21, wherein the controls include a temperature control configured to allow a user to select and set the target temperature.

25. The cooking appliance according to claim 21, wherein the controls include a timer control configured to allow a user to select the specified time for the shut-off timer.
